(19) United States
(12) Patent Application Publication
(10) Pub. No.: US 2023/0363712 A1
(43) Pub. Date: Nov. 16, 2023

(54) EQUINE TAIL SENSOR

(71) Applicant: Garmin International, Inc., Olathe, KS (US)

(72) Inventors: Bradley K. Smith, Cary, NC (US); Paul E. Earl, Apex, NC (US); Christopher R. Koszarsky, Holly Springs, NC (US)

(21) Appl. No.: 18/316,404

(22) Filed: May 12, 2023

Related U.S. Application Data
(60) Provisional application No. 63/364,683, filed on May 13, 2022.

(51) Int. Cl. A61B 5/00; A61B 5/024; A01K 29/00; G16H 40/63

(52) U.S. Cl. CPC A61B 5/6813; A61B 5/02427; A61B 5/02438; A61B 5/6831; A61B 5/0004; A01K 29/005; G16H 40/63; A61B 2503/40

(57) ABSTRACT

A heart rate monitor can comprise a heart rate sensor and a strap. The heart rate sensor can contact a tail of an equine and comprise an optical heart rate module (OHR) and a transmitter. The OHR module can output light into skin of the equine, receive reflected light, and convert the reflected light into electrical signals. The transmitter can wirelessly transmit heart rate data based on the electrical signals. The strap can removably receive the heart rate sensor and removably couple to the tail of the equine.

### RELATED APPLICATIONS

This application claims priority to U.S. provisional application 63/364,683 filed May 13, 2022, the contents of which are hereby incorporated by reference herein for all purposes.

### TECHNICAL FIELD

The present disclosure relates generally to an equine tail sensor.

### BACKGROUND

A heart rate of an animal can be measured via wired electrical sensors. These wired electrical sensors can be wrapped around an animal's torso and must be held against the animal's body to accurately detect electrical signals, from which various metrics may be calculated based on the detected electrical signals.

### BRIEF DESCRIPTION OF THE DRAWINGS

The detailed description references the accompanying figures. The use of the same reference numbers in different instances in the description and the figures may indicate similar or identical items. In addition, the proportion and the relative scale of the elements provided in the figures are intended to illustrate various embodiments of the present disclosure and are not to be used in a limiting sense.

FIG. 1 illustrates an example of an equine wearing a heart rate monitor device.

FIG. 2 illustrates an example of a user using a strap.

FIG. 3A is a top view of a heart rate sensor.

FIG. 3B is a bottom view of a heart rate sensor.

FIG. 3C is an isometric view of a heart rate sensor.

FIG. 4 is a top view of a heart rate sensor.

FIG. 5A is a bottom view of a heart rate monitor device.

FIG. 5B is a top view of a heart rate monitor device.

FIG. 6 is a block hardware diagram of a heart rate sensor.

FIG. 7 is an isometric view of a computing device.

FIG. 8 is a block hardware diagram of a heart rate monitor system.

### DETAILED DESCRIPTION

The present disclosure includes a heart rate monitor. The heart rate monitor can comprise a heart rate sensor and a strap. The heart rate sensor can contact a tail of an equine and comprise an optical heart rate module (OHR) and a transmitter. The OHR module can output light into skin of the equine, receive reflected light, and convert the reflected light into electrical signals. The transmitter can wirelessly transmit heart rate data based on the electrical signals. The strap can removably receive the heart rate sensor and removably couple to the tail of the equine. An equine can be a horse, pony, donkey, mule, or zebra, for example.

Conventional animal heart rate monitors use electrical sensors wrapped around an animal's torso, which often leads to inaccurate measurements. For example, electrical sensors can be coupled to a girth, which secures a saddle to an equine. When coupled to a girth, the electrical sensors can have discontinuities in the recorded data due to interference between the electrical sensors and the skin from the fur of the animal or vibration from the girth moving in response to the equine moving. Further, some heart rate monitors require an equine to be shaved where the sensors are placed and/or learn to move (e.g., walk, trot, canter, etc.) on a treadmill with wires strung from the equine to an electrocardiogram (EKG), for example.

The heart rate monitor disclosed herein is a heart rate monitor that is configured to provide accurate and complete heart rate data while easily and comfortably being worn by an equine. Although a heart rate monitor for an equine will be used as an example throughout this application, the heart rate monitor can be used on any tailed mammal and the heart rate monitor can measure and record various metrics including, but not limited to, heart rate. For example, the heart rate monitor can record a wide variety of metrics regarding the equine and its movement.

In various embodiments, the strap of the heart rate monitor has a cavity encased in plastic to receive the heart rate sensor. The strap can further include an attachment portion, for example a hook and loop fastener (e.g., Velcro), to couple the heart rate sensor to the tail of the equine in a manner that allows the heart rate sensor to be tightly held against the underside of the equine's tail.

The underside of an equine's tail is bare (e.g., hairless), which allows direct and repeatable contact with the skin. Direct and repeatable contact with the skin reduces discontinuities in the data and prevents the equine from needing to be shaved. The tail of an equine can remain relatively stationary compared to other body parts of the equine when the equine is sleeping, eating, grazing, walking, trotting, and/or cantering, for example. Fastening the heart rate monitor to a more stationary part of the equine can minimize vibration, which can allow the heart rate monitor to record data more accurately.

A user, for example, an owner, rider, veterinarian, boarder, and/or trainer, can fasten a heart rate monitor to an equine. Current heart rate monitors can be fastened in sensitive areas for some equines. For example, some heart rate monitors can be attached to or embedded in a girth. Due to interference caused by hair on the barrel of the equine where the girth is cinched, a user may overtighten the girth in an attempt to get more accurate sensor readings. This may cause pain or discomfort to the equine which could lead to behavioral issues, such as, ear pinning, body tensing, biting, kicking, and/or bucking. Over time the equine could become cinch-sensitive, which could prevent the horse from being saddled and/or ridden. In contrast, since the equine is hairless under the tail, the present heart rate monitor coupled to the tail of the equine can produce more accurate measurements, which can prevent the user from over tightening the strap in an attempt to get more accurate measurements.

In some examples, the heart rate monitor can include additional features to make wearing the heart rate monitor more comfortable for an equine. For example, the strap can be tapered along the edges to wrap evenly around the tail as the tail circumference decreases in size outwards from the equine body. Additionally, the strap can be elastic with openings to allow movement of vertebrae in the tail of the equine. Further, the strap can have textured neoprene that contacts the tail to prevent the strap from moving down the tail of the equine.

The heart rate monitor can also include features to make fitting the heart rate monitor to the equine safer and easier for the user. For example, the strap can include one or more cavities to receive a number of fingers or a hand. The cavities or other grips can allow a user to place and secure the strap around the tail of the equine and fasten the heart rate monitor one handed. One handed fitting enables the user to stand further away from the equine and minimizes the amount of time the user spends towards the rear of the equine, which is an area that can be dangerous if an equine kicks, for example.

In some examples, the heart rate sensor can include a button, a tab, and a light-emitting diode (LED). The button can power on and power off the heart rate sensor. The tab can consistently orient the heart rate sensor within the strap, as well as prevent the button from being accidentally pressed. The LED can emit a light to indicate whether the heart rate sensor is powered on. In a number of embodiments, the heart rate sensor can further include a processor, a memory, a wireless transmitter, a battery, an inertial sensor, a thermometer, a barometric sensor, and/or a location determining component.

In some configurations, the heart rate monitor is configured to work in combination with a computing device including a user's wearable device, mobile phone (e.g., smartphone), laptop, desktop, cloud device, hand-held portable computer, tablet computer, personal digital assistant, multimedia device, media player, and/or game device. For example, data recorded at the heart rate monitor can be wired or wirelessly transmitted to a computing device via a USB cable, Bluetooth, and/or a cellular network. Wireless data transmission enables an equine to wear the heart rate monitor on trails, during competitions, during training, when grazing, when trailering, and/or in a stall and allows a user to monitor an equine's metrics from a computing device near to or far from the equine.

The computing device can include a number of components, for example, a processor, a memory, a transmitter, a location determining component, a barometric sensor, and/or a user interface. The components of the computing device can be used to generate, analyze, and convey data. For example, the computing device can determine a heart rate, temperature, oxygen content, gait, and/or step count of an equine based on data received from the heart rate monitor and/or data generated at the computing device. The computing device can also use data generated by the computing device and/or the heart rate monitor to determine how long and/or how hard to work an equine. In a number of instances, the computing device can diagnose or alert a user that an equine may be ill or injured.

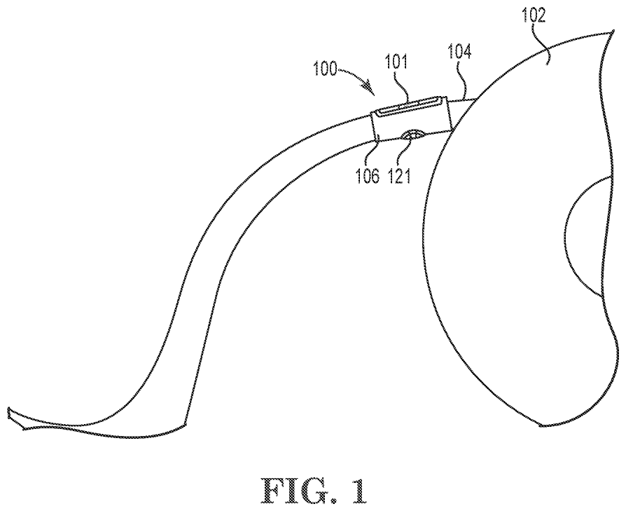

FIG. 1 illustrates an example of an equine 102 wearing a heart rate monitor 100. Heart rate monitor 100 is operable to record biometric and fitness information of an equine 102. The heart rate monitor 100 may be configured in a variety of ways. For instance, heart rate monitor 100 can be configured for use during equine competitions (e.g., eventing, hunter jumper, dressage, barrel racing, racing, trick riding, and/or rodeos), grazing, training, sleeping, resting, and/or trail riding.

As illustrated in FIG. 1, the heart rate monitor 100 includes a strap 106, which is shown in a looped (e.g., closed) configuration, wrapped around a tail 104 of the equine 102. The heart rate monitor 100 is removably coupled to a tail 104 of the equine 102 by the strap 106 circling the tail 104 of the equine 102. A portion of arrow 101 is included for reference, pointing toward the equine 102. Since the underside of an equine's tail 104 is void of hair, an OHR module 121 of the heart rate monitor 100 can directly contact the skin of the equine 102. Hair between the OHR module 121 and the skin of the equine 102 can prevent receivers of the OHR module 121 from receiving light, which can cause discontinuities in data recorded by the OHR module 121. Discontinuities in the data can cause the heart rate monitor 100 to provide stale (e.g., obsolete) and/or inaccurate data. Accordingly, direct contact of the OHR module 121 with the skin allows the heart rate monitor 100 to provide more accurate data in real-time.

Movement of receivers of the OHR module 121 and/or the body of the equine 102 can prevent the receivers of the OHR module 121 from receiving light, which can also cause discontinuities in the data. The barrel of the equine 102, which is where many current heart rate monitors are attached, can move with the inhaling and exhaling of the equine 102. Heart rate monitors at the barrel of the equine 102, often are attached to the equine 102 via a girth, which can attach to a saddle of the equine 102. The weight of the saddle and/or rider can cause the girth and the heart rate monitor to vibrate and/or move, which can prevent the receivers from receiving the light, which can also cause discontinuities in the data. A user may notice that the data is lagging or incorrect and try to tighten the girth in an attempt to get more accurate readings. Over cinching the girth may cause pain or discomfort to the equine 102 which could result in temporary or permanent physical or mental harm to the equine 102 and/or cause the equine 102 to react in a manner, which could be dangerous to a person.

The tail 104 of an equine 102 can remain relatively stationary compared to other body parts, for example, the barrel of the equine 102. Accordingly, attaching the heart rate monitor 100 to the tail 104 of the equine 102 can reduce exposure of the heart rate monitor 100 to movement and/or vibration and allows the receivers to receive light with fewer or zero interruptions. As such, the heart rate monitor 100 attached to the tail 104 of the equine 102 can more accurately record data than current heart rate monitors.

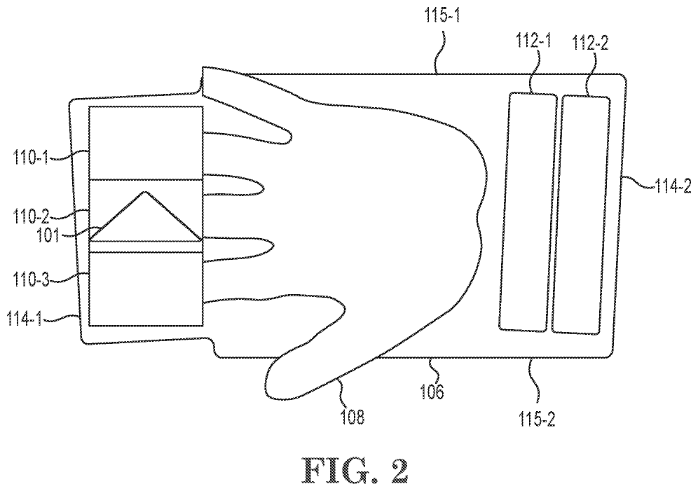

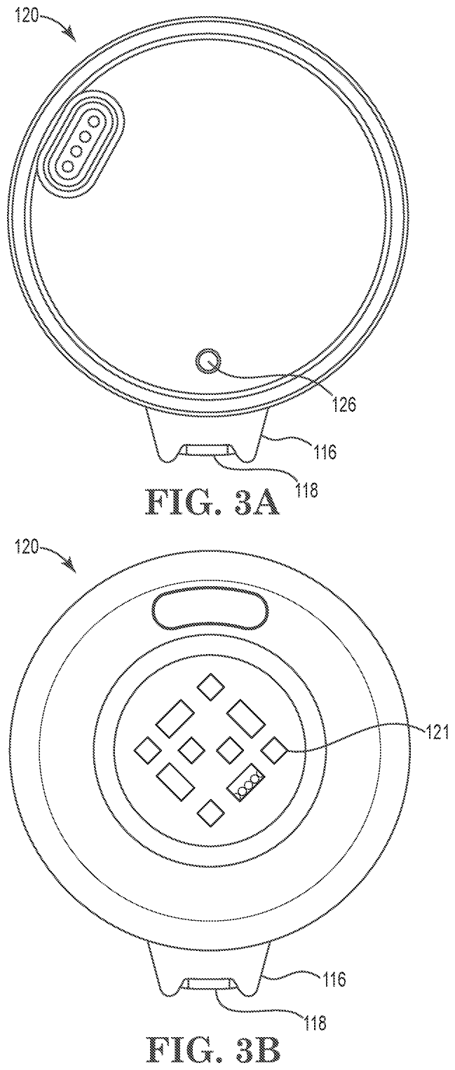

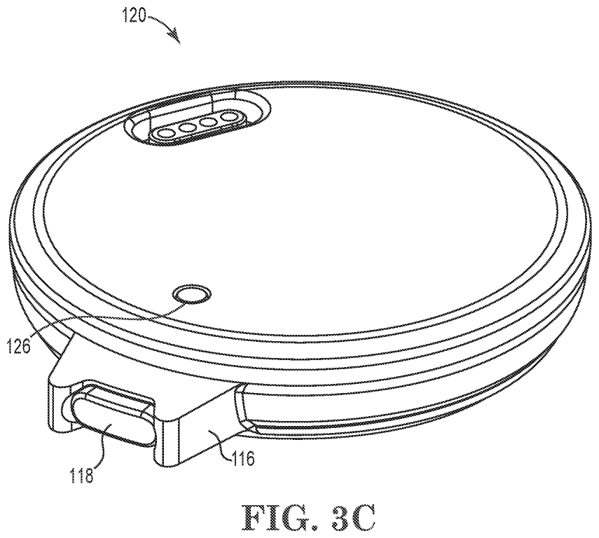

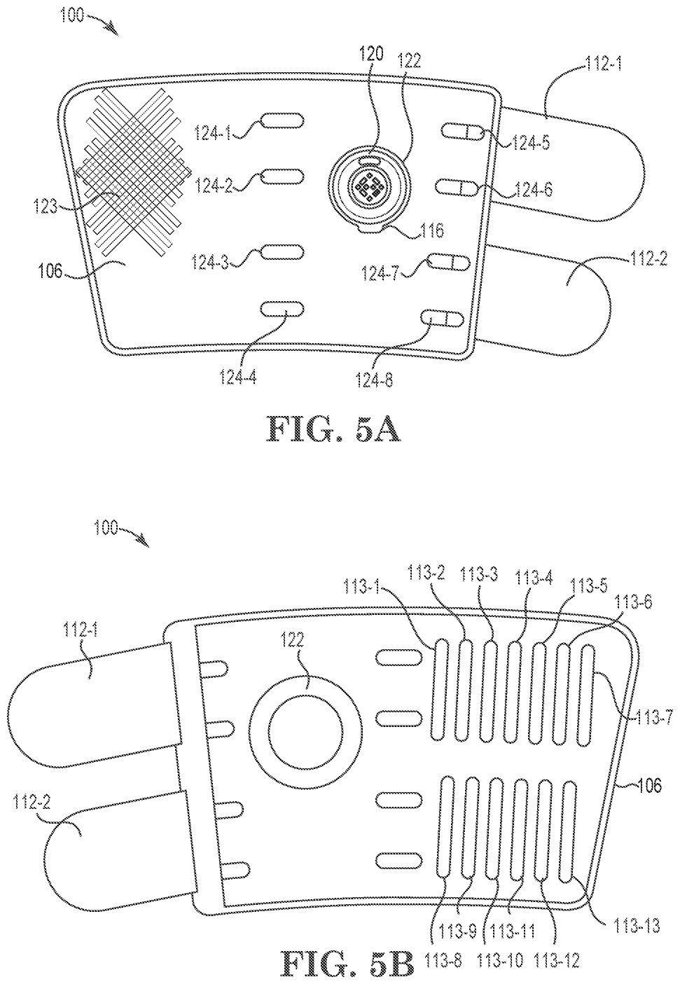

FIG. 2 illustrates an example of a user using a strap 106 of the heart rate monitor (e.g., heart rate monitor 100 in FIG. 1) while the strap 106 is in a non-looping (e.g., open) configuration ready to receive a tail (e.g., tail 104 in FIG. 1) of an equine (e.g., equine 102 in FIG. 1). The arrow 101 is illustrated in a same orientation as is illustrated in FIG. 1, such that it would be pointing toward the equine when attached thereto. The strap 106 is a strip of material configured to removably couple to a tail of an equine by looping around the tail. The strap 106 is configured to removably receive a heart rate sensor (e.g., heart rate sensor 120 in FIGS. 3A, 3B, 3C, 4, 5A, 6, 8). The heart rate sensor can be received by the strap 106 in a location under the hand 108 of the user (see, for example, FIGS. 5A-5B). The strap 106 can include or be made of elastic material to allow edges 115-1, 115-2 of the strap 106 to vary in length based on a tail size. In a number of embodiments, a length of edge 115-1 can correspond to a first circumference of a typical equine's tail and a length of edge 115-2 can correspond to a second circumference of a typical equine's tail.

In some examples, the strap 106 can taper, as illustrated by tapered edges 114-1, 114-2, from a larger circumference, represented by edge 115-1, to a smaller circumference, represented by edge 115-2. The taper is illustrated by the tapered edge 114-1 having a diagonal orientation with respect to the vertical (as oriented in FIG. 2) and the tapered edge 114-2 having an opposite diagonal orientation such that the two tapered edges 114-1, 114-2 taper toward each other when the strap 106 is in an open position, as illustrated in FIG. 2. The tapered edges 114-1, 114-2 make the strap 106 form an open ended cone (rather than a cylinder) when the strap 106 is in a closed configuration, such as when attached to the tail of the equine. A circumference of an equine's tail gets gradually smaller from a dock of the tail to an end of the tail. The strap 106 includes the tapered edges 114-1, 114-2 to properly fit the tail circumference decreasing over the length of the tail. The tapered edges 114-1, 114-2 can also help prevent the strap 106 from moving and/or make the strap 106 more comfortable for the equine to wear because it is evenly following the circumference of the tail.

The strap 106 can include a number of attachment portions 112-1, 112-2 to couple the heart rate sensor to the tail of the equine in a manner that allows an OHR module (e.g., OHR module 121 in FIG. 1) to be tightly held against the underside of the equine's tail. The number of attachment portions 112-1, 112-2 can be, but are not limited to, strips of material having a hook or loop fastener thereon, where an opposite of the hook or loop fastener is on an underside portion of the strap 106 opposite cavities 110-1, 110-2, 110-3 so that the strap 106 can be looped and secured to itself in a closed configuration. Other examples of the attachment portions 112-1, 112-2 can be a clips, magnets, and/or snaps, among others.

The strap 106 can include a number of cavities 110-1, 110-2, 110-3 to receive a portion of a number of fingers and/or a portion of a hand 108 of a user. In some examples, as illustrated in FIG. 2, cavity 110-1 can receive a portion of a first finger, cavity 110-2 can receive a portion of a second finger, and cavity 110-3 can receive a portion of a third finger. This allows a user to quickly wrap the strap 106 around a tail of an equine and fasten the heart rate monitor one handed. Without the number of cavities 110-1, 110-2, 110-3, a user would likely need to use both hands to fasten the strap 106 to the tail of the equine. Using both hands would position the user closer to the equine because both hands would need to be stretched out towards the equine with the user's chest, stomach, and both legs exposed to the equine. One handed fitting enables the user to face towards the head of the equine to watch for signs of agitation like ear-pinning or relaxation like a lowered head, licking, and/or chewing while fastening the strap 106.

Often equines are trained to be approached and tacked on their left side. Accordingly, in a number of embodiments, the number of cavities 110-1, 110-2, 110-3 can be designed to receive a right hand of a user to enable the user to approach the left side of an equine and fasten the strap 106 to a tail of the equine by placing their hand with the open strap 106 under the tail and closing their hand with the strap 106 to loop the strap 106 around the tail while facing towards the head of the equine to monitor the equine's disposition.

FIG. 3A is a top view of a heart rate sensor 120, FIG. 3B is a bottom view of the heart rate sensor 120, and FIG. 3C is an isometric view of the heart rate sensor 120. The heart rate sensor 120 can contact a tail (e.g., tail 104 in FIG. 1) of an equine (e.g., equine 102 in FIG. 1).

The heart rate sensor 120 can include a tab 116, a button 118, an LED 126, and/or an OHR module 121. The tab 116 can consistently orient the heart rate sensor 120 within a strap (e.g., strap 106 in FIGS. 1-2), as will be further discussed in connection with FIGS. 5A-5B. The tab 116 can also act as a button guard to prevent the button 118 from being accidentally pressed while installed. For example, the tab 116 can include a cavity that houses the button 118, which covers a portion of the button 118 to prevent the button 118 from being pressed while the heart rate sensor 120 is attached to an equine.

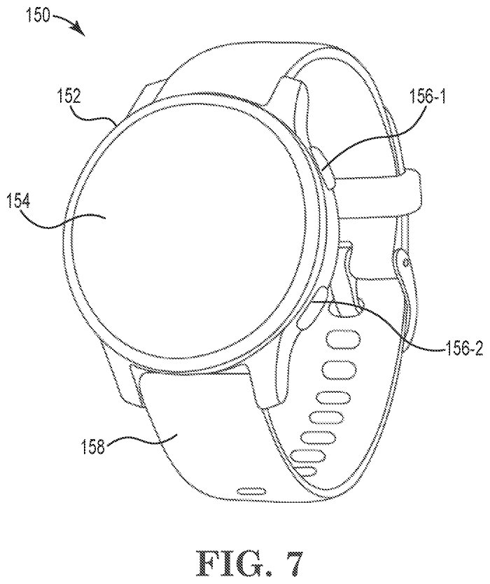

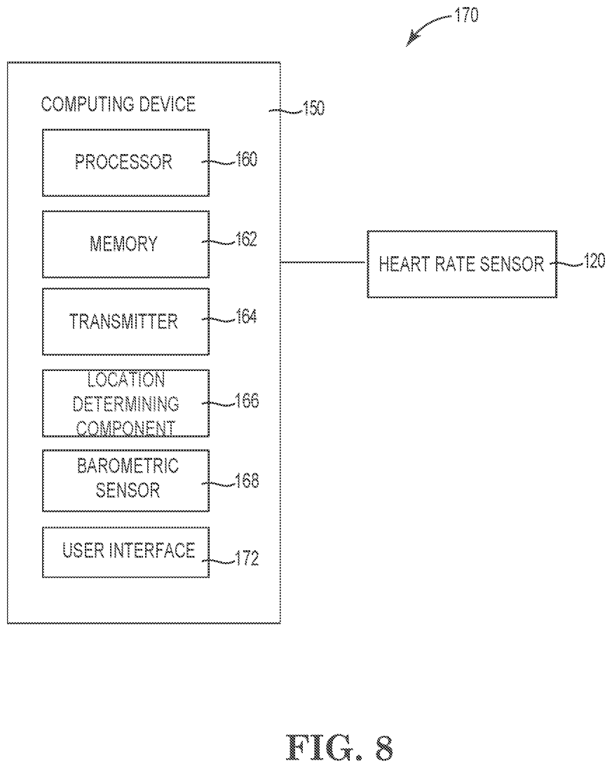

The button 118 can power on (e.g., turn on) and power off (e.g., turn off) the heart rate sensor 120. A user can know when the heart rate sensor 120 is powered on or powered off based on the LED 126. For example, the LED 126 can emit a light to indicate the heart rate sensor 120 is powered on or the LED 126 can emit a particular color light or flashing pattern to indicate the heart rate sensor 120 is powered on. In some embodiments, the LED 126 can indicate an issue, for example, a low battery, an error with the heart rate sensor 120, and/or a connectivity issue between the heart rate sensor 120 and a computing device (e.g., computing device 150 in FIGS. 7-8) by emitting a particular color light or flashing pattern.

The OHR module 121 can include emitters (e.g., LEDs) to transmit visible and/or non-visible light and receivers (e.g., photodiodes) to receive (e.g., detect) visible and/or non-visible light that generate a light intensity signal based on the received reflection of light. The received light can be converted into an electrical signal, including, but not limited to a current. Conversion can include but is not limited to the generation of digital data corresponding to the received light, which may include filtering, calculations, and other manipulations of data corresponding to the received light. The electrical signal can be converted into a digital value by an analog to digital converter.

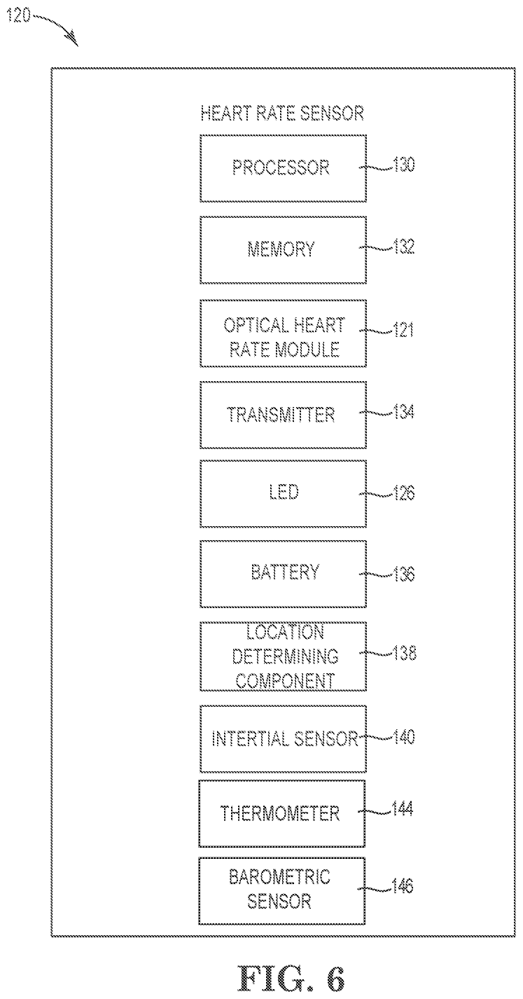

Each LED can generate light based on an intensity determined by a processor (e.g., processor 130 in FIG. 6). For example, LEDs may include any combination of green LEDs, red LEDs, and/or infrared or near infrared LEDs that may be configured by the processor to emit light into an equine's skin. In some embodiments, the red LEDs operate at a wavelength between approximately 610 and 700 nanometers (nm). In some embodiments, a first LED produces light at approximately 630 nm, a second LED operates at approximately 940 nm, and a third LED operates at approximately 660 nm.

The OHR module 121 also includes one or more photodiodes capable of receiving transmissions or reflections of visible-light and/or infrared (IR) light output by the LEDs into the equine's skin. The OHR module 121 can generate a pulse oximeter (SpO2) signal based on an intensity of the reflected light received by each photodiode. The light intensity signals generated by the one or more photodiodes may be communicated to the processor. In a number of embodiments, the processor can include an integrated photometric front end for signal processing and digitization. In other embodiments, the processor is coupled to a separate photometric front end. The photometric front end may include filters for the light intensity signals and analog-to-digital converters to digitize the light intensity signals into SpO2 signals including a cardiac signal component associated with the equine's heartbeat.

Typically, when the OHR module 121 is worn against the equine's body, the one or more emitters are positioned against the equine's skin to emit light into the skin and the one or more receivers are positioned near the emitters to receive light emitted by the one or more emitters after transmission through or reflection from the skin. The alignment of the emitters and receivers can suitably measure the equine's heart rate when the equine is in motion or moving its tail. The processor may receive a SpO2 signal based on a light intensity signal output by one or more receivers based on an intensity of light after transmission of the light through or reflection from the skin that has been received by the receivers.

In both the transmitted and reflected uses, the intensity of measured light may be modulated by a cardiac cycle due to variation in tissue blood perfusion during the cardiac cycle. In activity environments, the intensity of measured light may also be strongly influenced by many other factors, including, but not limited to, static and/or variable ambient light intensity, body motion at measurement location, static and/or variable sensor pressure on the skin, motion of the sensor relative to the body at the measurement location, breathing, and/or light barriers (e.g., hair, opaque skin layers, sweat, etc.). Relative to these sources, the cardiac cycle component of the SpO2 signal can be very weak, for example, by one or more orders of magnitude.

The electrical signals converted from received light can be used to determine cardiac information including heart rate and/or pulse oximetry data. The electrical signals and/or cardiac information including the heart rate data and/or pulse oximetry data can be transmitted to, for example, a computing device (e.g., computing device 150 in FIGS. 7-8).

In a number of embodiments, the heart rate data can be used to determine (e.g., calculate) heart rate, heart rate variability, respiration rate, stress level, and/or resting heart rate at the heart rate sensor 120 or at the computing device. A rate of change of the heart rate can be determined by tracking peak to peak of the heart rate, which is a photoplethysmography (PPG) heart rate. The rate of change of the heart rate can be used to diagnose disease and/or illness including colic and the flu, for example.

The heart rate data can further be used to establish sleep metrics and/or a body battery for an equine. For example, sleep stages and/or sleep quality can be based on an equine's heart rate and/or the rate of change of the heart rate. The body battery can determine whether an equine is ready to take on a particular activity based on their heart rate and/or the rate of change of their heart rate. The heart rate sensor 120 and/or the computing device can compare the heart rate to an equine's resting heart rate range around 20-30 beats per minute (BPM), an equine's heart rate during high intensity around 300 BPM, and/or a heart rate variability, which can be up to 20 BPM per second. Since an equine's heart rate can change very rapidly due to emotion and environmental cues, motion (e.g., accelerometer data) is not a reliable indicator of heart rate change.

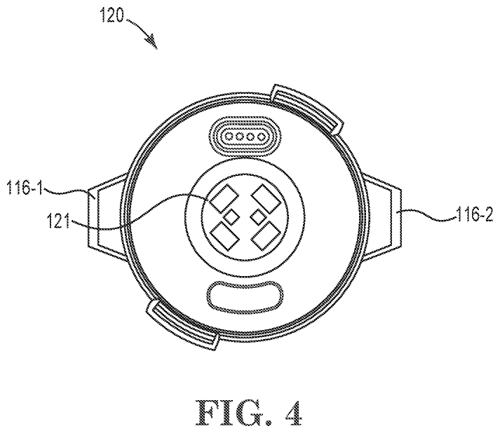

FIG. 4 is a top view of a heart rate sensor 120. The heart rate sensor 120 can include an OHR module 121 and a number of tabs 116-1, 116-2. The number of tabs 116-1, 116-2 can consistently orient the heart rate sensor 120 within a strap (e.g., strap 106 in FIGS. 1-2), as will be further discussed in connection with FIGS. 5A-5B. The tabs 116-1, 116-2 can also prevent a button (e.g., button 118 in FIGS. 3A-3C) or a number of buttons from being accidentally pressed. For example, tab 116-1 can include a cavity that houses the button, which covers a portion of the button.

FIG. 5A is a bottom view of a heart rate monitor 100 and FIG. 5B is a top view of the heart rate monitor 100. The heart rate monitor 100 can include heart rate sensor 120 and strap 106.

The strap 106 can include a cavity 122 to receive the heart rate sensor 120. The heart rate sensor 120 can be pressed against skin of an equine (e.g., equine 102 in FIG. 1) by the strap 106 to enable accurate generation of health metrics. The cavity 122 can partially enclose the heart rate sensor 120 and protect the heart rate sensor 120 from environmental factors including water and debris. In some instances, the cavity 122 can be encased in plastic to prevent the heart rate sensor 120 from being damaged. The plastic can also create a number of rigid surfaces to maintain the heart rate sensor 120 within the cavity 122 and prevent the heart rate sensor 120 including tab 116 from rotating. The cavity 122 can be made from thermoplastic polyurethane (TPU), for example.

The tab 116 can consistently orient the heart rate sensor 120 within the strap 106. For example, the tab 116 can extend outward from the body of the heart rate sensor 120 towards an end of a tail (e.g., tail 104 in FIG. 1) of an equine. This allows the heart rate sensor 120 and/or a computing device (e.g., computing device 150 in FIGS. 7-8) to know a direction of movement of the heart rate sensor 120.

In a number of embodiments, the cavity 122 can removably receive the heart rate sensor 120. This enables the strap 106 to be washed with the heart rate sensor 120 removed and the heart rate sensor 120 to be utilized with different straps. The different straps can be clean straps and/or different size straps. The heart rate sensor 120 is inserted on the side of the heart rate monitor 100 shown in FIG. 5A to provide a continuous, seamless, smooth, and/or soft surface on the side of the heart rate monitor 100 shown in FIG. 5B that contacts the main body of an equine to prevent irritation on the main body of the equine.

The strap 106 can include an attachment portion 112-1, 112-2 to couple the heart rate monitor 100 to a tail of an equine. The attachment portion 112-1, 112-2 can be a hook and loop fastener, a clip, a magnet, and/or a snap. For example, the attachment portion 112-1, 112-2 can be Velcro or any material that fastens to Velcro and size indicators 113-1, 113-2, 113-3, 113-4, 113-5, 113-6, 113-7, 113-8, 113-9, 113-10, 113-11, 113-12, 113-13 can be Velcro or any material that fastens to Velcro. The strap 106 can create a loop around a tail of an equine when the attachment portion 112-1, 112-2 is coupled to one or more of the number of size indicators 113-1, . . . , 113-13.

The size indicators 113-1, . . . , 113-13 can mark how tight the strap 106 is attached to a tail of an equine. A user can make a note of which size indicator of the number of size indicators 113-1, . . . , 113-13 the attachment portion 112-1, 112-2 is attached to. That way, if the user decides the strap 106 is too tight or too loose, the user knows which size indicator of the number of size indicators 113-1, . . . , 113-13 to adjust from. If the heart rate monitor 100 appears to be getting accurate readings and the strap 106 is comfortable for the equine, the user can continue to use the same size indicator of the number of size indicators 113-1, . . . , 113-13.

FIG. 5A illustrates the side of the heart rate monitor 100 that contacts a tail of an equine. The strap 106 can be elastic with a number of openings 124-1, 124-2, 124-3, 124-4, 124-5, 124-6, 124-7, 124-8. The elastic and/or the openings 124-1, . . . , 124-8 can allow movement of vertebrae in a tail of an equine.

Further, a portion of the strap 106 can include a textured feature 123. For example, a portion or the entire side of the strap 106 illustrated in FIG. 5A can include the textured feature 123. The textured feature 123 can grip to a tail by providing a greater coefficient of friction between the textured feature 123 and the tail than between the material forming the strap 106 and the tail. The textured feature 123 can prevent the strap 106 from moving down the tail of the equine. In a number of embodiments, the textured feature 123 can be neoprene.

FIG. 5B illustrates the opposite side of the heart rate monitor 100 illustrated in FIG. 5A. Fabric of the strap 106 can be continuous, seamless, smooth, and/or soft across the backside of the cavity 122. At times the side of the strap 106 illustrated in FIG. 5B including the backside of the cavity 122 which extrudes out from the strap 106, may come into contact with a body and/or anus of an equine. As previously discussed, having fabric continuous, seamless, smooth, and/or soft across the strap 106 including the backside of the cavity 122, can prevent irritation to an equine wearing the heart rate monitor 100. The fabric can be stretchable fabric or a non-stretchable material such as leather or koskin (e.g., faux leather), for example.

FIG. 6 is a block hardware diagram of a heart rate sensor 120. The heart rate sensor 120 includes a processor 130, a memory 132, an OHR module 121, a transmitter 134, an LED 126, a battery 136, a location determining component 138, an inertial sensor 140, a thermometer 144, and/or a barometric sensor 146.

The processor 130 provides processing functionality for the heart rate sensor 120 and can include any number of processors, micro-controllers, circuitry, field programmable gate array (FPGA) or other processing systems, and resident or external memory for storing data, executable code, and other information. The processor 130 can execute one or more software programs embodied in a non-transitory computer readable medium (e.g., memory 132) that implement techniques described herein including outputting light into skin of an equine (e.g., equine 102 in FIG. 1), receiving reflected light, converting the reflected light into electrical signals via OHR module 121 and wirelessly transmitting the heart rate data based on the electrical signals via transmitter 134. The processor 130 is not limited by the materials from which it is formed or the processing mechanisms employed therein and, as such, can be implemented via semiconductor(s) and/or transistors (e.g., using electronic integrated circuit (IC) components), and so forth.

The memory 132 can be a tangible, computer-readable storage medium that provides storage functionality to store various data and/or program code associated with an operation, such as software programs and/or code segments, or other data to instruct the processor 130, and possibly other components of the heart rate sensor 120, to perform the functionality described herein. The memory 132 can store data, such as program instructions for operating the heart rate sensor 120 including its components, and so forth. The memory 132 can also store heart rate data, temperature data, oxygen content, gait, step count, geographic location data, speed data, pace data, cadence data, distance traveled data, calories burned data, and the like.

It should be noted that while a single memory 132 is described, a wide variety of types and combinations of memory (e.g., tangible, non-transitory memory) can be employed. The memory 132 can be integral with the processor 130, can comprise stand-alone memory, or can be a combination of both. Some examples of the memory 132 can include removable and non-removable memory components, such as random-access memory (RAM), read-only memory (ROM), flash memory (e.g., a secure digital (SD) memory card, a mini-SD memory card, and/or a micro-SD memory card), magnetic memory, optical memory, universal serial bus (USB) memory devices, hard disk memory, external memory, and so forth. In a number of embodiments, the heart rate sensor 120 and/or the memory 132 can include removable integrated circuit card (ICC) memory, such as memory provided by a subscriber identity module (SIM) card, a universal subscriber identity module (USIM) card, a universal integrated circuit card (UICC), and so on.

The transmitter 134 can receive control signals and/or other communications from, for example, a computing device (e.g., computing device 150 in FIGS. 7-8). The transmitter 134 can be communicatively coupled to a computing device via a wired or wireless connection. Accordingly, the transmitter 134 can be a wireless transmitter configured to transmit data, including heart rate data and/or sensor data, to a computing device via Bluetooth and/or a cellular network, for example.

The LED 126 can be included in the heart rate sensor 120, such that when turned on, the LED 126 is visible to a user. The LED 126 can be turned on, emit a particular color, and/or flash a particular light pattern to indicate to a user that the heart rate sensor 120 is on or off, the heart rate sensor 120 is or is not wirelessly coupled to a computing device, the OHR module 121 is or is not receiving light, the heart rate sensor 120 is or is not recording data, the heart rate sensor 120 is or is not functioning properly, and/or a battery life of the heart rate sensor 120.

The battery 136 and/or any other power source can be used to power one or more components of the heart rate sensor 120. In a number of embodiments, the battery 136 can be a coin cell. In some examples, the battery 136 can be or can include a thermoelectric generator to convert heat from the equine's body into electricity to power the heart rate sensor 120.

The heart rate sensor 120 may include a number of sensors for detecting an orientation, change in orientation, direction, change in direction, position, and/or change in position of the heart rate sensor 120. For example, the heart rate sensor 120 may include an inertial sensor 140 configured to detect an orientation, change in orientation, direction, change in direction. The inertial sensor 140 can be a gyroscope and/or an accelerometer.

In some embodiments, the inertial sensor 140 may incorporate one or more accelerometers positioned to determine the acceleration and direction of movement of the heart rate sensor 120. The accelerometer may determine magnitudes of acceleration in an X-axis, a Y-axis, and a Z-axis to measure the acceleration and direction of movement of the inertial sensor 140 in each respective direction (or plane). It will be appreciated by those of ordinary skill in the art that a three-dimensional vector describing a movement of the heart rate sensor 120 through three-dimensional space can be established by combining the outputs of the X-axis, Y-axis, and Z-axis accelerometers using known methods. Single and multiple axis models of the inertial sensor 140 are capable of detecting magnitude and direction of acceleration as a vector quantity and may be used to sense orientation and/or coordinate acceleration of an equine.

The heart rate sensor 120 can also include a location determining component 138 (e.g., position determining component) that is configured to detect a position measurement for the heart rate sensor 120 (e.g., geographic coordinates of at least one reference point on the heart rate sensor 120). In at least one embodiment, the location determining component can be a GNSS receiver (e.g., a global positioning system (GPS) receiver, assisted-GPS, software defined (e.g., multi-protocol) receiver, or the like). The location determining component 138 generally determines a current geolocation of the heart rate sensor 120 and may process a first electronic signal, such as radio frequency (RF) electronic signals, from a global navigation satellite system (GNSS) such as the global positioning system (GPS) primarily used in the United States, the GLONASS system primarily used in the Soviet Union, or the Galileo system primarily used in Europe. The location determining component 138 may include satellite navigation receivers, processors, controllers, other computing devices, or combinations thereof, and memory. The location determining component 138 may be in electronic communication with an antenna (not shown) that may wirelessly receive an electronic signal from one or more of the previously-mentioned satellite systems and provide the electronic signal to location determining component 138. The location determining component 138 may process the electronic signal, which includes data and information, from which geographic information such as the current geolocation is determined. The current geolocation may include geographic coordinates, such as the latitude and longitude, of the current geographic location of the heart rate sensor 120. The location determining component 138 may communicate the current geolocation to the processor 130. Generally, the location determining component 138 is capable of determining continuous position, velocity, time, and direction (heading) information.

The number of sensors of the heart rate sensor 120 can further include thermometer 144 and/or barometric sensor 146. The thermometer 144 can measure a temperature under a tail of an equine. The barometric sensor 146 can be used to determine an elevation and/or grade of a slope.

Sensor data generated from the number of sensors can be wired or wirelessly transmitted and used to determine sleep quality, diagnose illness, and/or track physical activity of an equine. For example, if the inertial sensor 140 records a number of 360 degree rotations of an equine the heart rate sensor 120 can determine the equine has rolled a number of times. The heart rate sensor can determine that the equine may be suffering from colic since rolling can be a symptom of colic. The heart rate sensor 120 can alert a user to the equine's possible malady or can wait for a threshold number of symptoms that also suggest colic prior to notifying a user.

FIG. 7 is an isometric view of a computing device 150. The computing device 150 can be a cloud device, a remote server, a tablet, a laptop, a desktop computer, a smartphone, and/or a wearable electronic device such as a smartwatch. The computing device 150 can be paired with a heart rate monitor (e.g., heart rate monitor 100 in FIGS. 1, 5A, 5B) and/or a heart rate sensor (e.g., heart rate sensor 120 in FIGS. 3A, 3B, 3C, 4, 5A, 5B, 6, 8) to receive data therefrom. The computing device 150 can receive data, including heart rate data, from the heart rate sensor. In a number of embodiments, the computing device 150 can store the data in memory and/or convey the received data to a user via a user interface and/or a speaker. For example, the computing device 150 can present information based on physiological characteristics including heart rate, heart rate variability, blood pressure, and/or SpO2 percentage or a physiological response including stress level and/or body battery (e.g., body energy level) of an equine (e.g., equine 102 in FIG. 1).

When the computing device 150 is a smartwatch, as illustrated in FIG. 7, the computing device 150 can include a band 158 coupled to a housing 152 including a number of buttons 156-1, 156-2 and a display 154. The buttons 156-1, 156-2 are configured to control a number of functions of the computing device 150. Such a configuration is useful, for example, when the user is riding the equine. The rider may monitor the performance of the equine (e.g, heart rate, stress, temperature, gait, and/or other metrics) in real time while riding and remaining in control of the equine, without having to wait for post-exercise analysis of the equine's performance. The computing device 150 may include audible feedback for the user, such as where the computing device 150 itself includes earpods or other speakers that may be heard by the user while riding or otherwise exercising the equine. In some configurations, the computing device 150 (e.g., smartwatch) may pair with earpods or other speakers to provide visual and audible feedback to the user.

Riders can utilize a horse's heart rate as a valuable indicator to optimize their riding experience and ensure the well-being of their equine partner. By monitoring the heart rate, a rider can gauge the equine's level of exertion and make informed decisions about pace and intensity. For instance, when the heart rate is elevated, the rider may opt to slow down or take a break, allowing the equine to recover and prevent overexertion. Additionally, paying attention to a equine's heart rate can help riders identify if the equine is struggling with a particular exercise or gait, providing valuable insights into areas that may require additional training or conditioning.

Furthermore, monitoring a equine's heart rate can be an essential tool in detecting and managing stress, ensuring a healthy and happy partnership between equine and rider. A consistently high heart rate, even during low-intensity activities, may signal that the equine is experiencing anxiety or discomfort, prompting the rider to investigate potential causes and take appropriate action. By regularly observing their equine's heart rate, riders can develop a deeper understanding of the animal's unique physical and emotional needs, ultimately leading to a more harmonious and successful riding experience. By adapting their riding style based on the equine's heart rate, riders can not only enhance their own skills, but also prioritize the well-being of their equine companion.

Incorporating geographic information, such as GPS track logs, into the analysis of equine performance metrics and heart rate data can further enhance the understanding of how environmental factors and terrain impact the animal's performance. By overlaying GPS data with heart rate and accelerometer information, users can identify specific locations or conditions where the equine's physiological responses or movement patterns change significantly.

For example, analyzing the combined data set may reveal that the equine's heart rate increases more than expected when climbing hills or navigating uneven terrain. This insight could indicate the need to adjust the training program to focus on improving strength and endurance in such conditions. Similarly, correlating GPS track logs with performance metrics may uncover variations in speed or gait patterns that occur in certain environments, such as sand, grass, or hard ground. Understanding these correlations can help riders and trainers tailor training programs to address any weaknesses or inconsistencies in the equine's performance across various terrains.

Moreover, the integration of geographic information can also assist in evaluating the impact of weather conditions, such as temperature and humidity, on the equine's heart rate and performance. This knowledge can be invaluable for planning training schedules and competition strategies that account for the influence of environmental factors on the equine's well-being and success.

The band 158 may be removably secured to the housing 152 via attachment of securing elements to corresponding connecting elements. Examples of securing elements and/or connecting elements include, but are not limited to hooks, latches, clamps, snaps, and the like. The band 158 may be made of a lightweight and resilient thermoplastic elastomer and/or a fabric, for example, such that the band 158 may encircle a portion of a user without discomfort while securing the housing 152 to the user. The band 158 may be configured to attach to various portions of a user, such as a user's leg, waist, wrist, forearm, and/or upper arm.

The display 154 may include a liquid crystal display (LCD), a thin film transistor (TFT), an LED, a light-emitting polymer (LEP), and/or a polymer light-emitting diode (PLED). However, embodiments are not so limited. The display 154 may be capable of displaying text and/or graphical information. The display 154 may be provided with a touch screen to receive input (e.g., data, commands, etc.) from a user. For example, a user may operate the computing device 150 by touching the touch screen and/or by performing gestures on the display 154. In some embodiments, the display 154 may be a capacitive touch screen, a resistive touch screen, an infrared touch screen, or any combinations thereof.

FIG. 8 is a block hardware diagram of a heart rate monitor system 170. The heart rate monitor system 170 can include a heart rate sensor 120 wirelessly coupled to a computing device 150. The computing device 150 can include a processor 160, a memory 162, a transmitter 164, a location determining component 166, a barometric sensor 168, and/or a user interface 172.

The processor 160 provides processing functionality for the computing device 150 and can include any number of processors, micro-controllers, circuitry, FPGA or other processing systems, and resident or external memory for storing data, executable code, and other information. The processor 160 can execute one or more software programs embodied in a non-transitory computer readable medium (e.g., memory 162) that implement techniques described herein including receiving heart rate data via transmitter 164. The processor 160 is not limited by the materials from which it is formed or the processing mechanisms employed therein and, as such, can be implemented via semiconductor(s) and/or transistors, and so forth.

The one or more processors 160 may be adapted and configured to execute any of a number of software applications and/or any of a number of software routines residing in memory 162, in addition to other software applications. One of the number of applications may be a client application that may be implemented as a series of machine-readable instructions for performing the various functions associated with implementing the performance of the heart rate monitor system 170 as well as receiving information at, displaying information on, and transmitting information from the computing device 150. The client application may function to implement a system wherein the front-end components communicate and cooperate with back-end components. The client application may include machine-readable instructions for implementing a user interface 172 to allow a user to input commands to, and receive information from, the computing device 150. One of the plurality of applications may be a native web browser, such as Apple's Safari®, Google Android™ mobile web browser, Microsoft Internet Explorer® for Mobile, Opera Mobile™, that may be implemented as a series of machine-readable instructions for receiving, interpreting, and displaying web page information from a server device or other back-end components while also receiving inputs from the computing device 150. Another application of the plurality of applications may include an embedded web browser that may be implemented as a series of machine-readable instructions for receiving, interpreting, and displaying web page information from the server device or other back-end components within the client application.

The client applications or routines may include an accelerometer routine that determines the acceleration and direction of movements of the heart rate sensor 120, which correlate to the acceleration, direction, and movement of the equine. The accelerometer routine may receive and process data from an inertial sensor (e.g., inertial sensor 140 in FIG. 6) to determine one or more vectors describing the motion of the equine for use with the client application. In some embodiments where the inertial sensor includes an accelerometer having X-axis, Y-axis, and Z-axis accelerometers, the accelerometer routine may combine the data from each accelerometer to establish the vectors describing the motion of the equine through three-dimensional space. In some embodiments, the accelerometer routine may use data pertaining to less than three axes.

The client applications or routines may further include a velocity routine that coordinates with the location determining component 166 of the computing device and/or the location determining component (e.g., location determining component 138 in FIG. 6) of a heart rate sensor (e.g., heart rate sensor 120 in FIG. 6) to determine or obtain velocity and direction information for use with one or more of the plurality of applications, such as the client application, or for use with other routines.

The user may also launch or initiate any other suitable user interface application to access a server device to implement an equine monitoring process. Additionally, a user may launch the client application from the computing device 150 to access the server device to implement the equine monitoring process.

After the above-described data has been gathered or determined by the sensors of computing device 150 and/or the heart rate sensor 120 and stored in memory 162, the heart rate sensor 120 and/or the computing device 150 may transmit information associated with measured cardiac information, such as heart rate (HR), pulse oximetry (SpO2) information, blood oxygen saturation percentage (pulse oximetry signal), peak-to-peak interval (PPI), heart-rate variability (HRV), motion data (acceleration information), location information, stress intensity level, and body energy level of an equine to computing device 150 and/or a server device for storage and additional processing. For example, the computing device 150 and/or a server may perform one or more processing functions remotely that may otherwise be performed by a heart rate monitor (e.g., heart rate monitor 100 in FIGS. 1, 5A, 5B). In such embodiments, the computing device 150 or server may include a number of software applications capable of receiving equine information gathered by the sensors to be used in determining a physiological response (e.g., a stress level, an energy level, etc.) of the equine. For example, the computing device 150 and/or the heart rate sensor 120 may gather information from sensors as described herein, but instead of using the information locally, the heart rate sensor 120 may send the information to the computing device 150 or the server for remote processing.

Through the application of data analytics and visualization tools, the stored heart rate data can be transformed into meaningful insights, revealing patterns and trends related to the equine's overall fitness, training progress, and response to stress. For example, by tracking changes in resting and peak heart rates over time, users can evaluate the efficacy of training programs and adjust them accordingly to ensure optimal performance. Additionally, analysis of heart rate variability (HRV) can serve as a valuable tool for assessing the equine's stress levels, allowing for early detection and intervention of potential health issues or behavioral problems. By integrating other data points, such as exercise regimens, competition results, and medical records, users can gain a comprehensive understanding of the interplay between various factors affecting the equine's health and performance. This information can be invaluable for making informed decisions on training adjustments, identifying potential health risks, and creating tailored plans to enhance the equine's well-being and performance in the long run.

Integrating stored equine heart rate data with additional performance metrics, such as accelerometer data, can offer a wealth of insights into the relationship between physiological responses and the animal's performance. Accelerometers, typically embedded in wearable devices, can measure various aspects of an equine's movement, including speed, stride length, and gait patterns. By correlating heart rate information with accelerometer data, users can identify potential areas for improvement, such as efficiency in specific gaits or response to different training methods.

For instance, a combined analysis of heart rate and accelerometer data can reveal the equine's level of exertion during specific exercises, offering insights into its fitness and adaptability. Users can observe how efficiently the equine maintains its heart rate at different speeds or during transitions between gaits, helping to fine-tune training programs and optimize performance. Additionally, by examining the data collected over time, it becomes possible to detect subtle changes in the equine's movement patterns or heart rate variability that may indicate the onset of fatigue or a developing health issue. By harnessing the power of integrated data analysis, riders, trainers, and veterinarians can make more informed decisions about an equine's training regimen, monitor progress, and ensure the animal's long-term health and well-being.

The computing device 150 or the server may perform the analysis of the gathered equine information to determine a stress level or a body energy level of the equine, for example. The server may also transmit information associated with the physiological response, such as a stress level, an energy level, of the equine. For example, the information may be sent to the server device and include a request for analysis, where the information determined by the server device is returned to computing device 150 and/or heart rate sensor 120. Such functionality may be useful for example, when the equine is being transported via a trailer. The user can remotely monitor the equine's heart rate, temperature, and other health metrics from the vehicle cabin to determine if the equine is stressed.

The memory 162 can be a tangible, computer-readable storage medium that provides storage functionality to store various data and/or program code associated with an operation, such as software programs and/or code segments, or other data to instruct the processor 160, and possibly other components of the computing device 150, to perform the functionality described herein. The memory 162 can store data, such as program instructions for operating the computing device 150 including its components, and so forth. The memory 162 can also store heart rate data, temperature data, oxygen content, gait, step count, geographic location data, speed data, pace data, cadence data, distance traveled data, calories burned data, and the like. In a number of embodiments, the memory 162 can store an application, which enables a user to view and analyze data received from the heart rate sensor 120 and/or the computing device 150.

It should be noted that while a single memory 162 is described, a wide variety of types and combinations of memory can be employed. The memory 162 can be integral with the processor 160, can comprise stand-alone memory, or can be a combination of both. Some examples of the memory 162 can include removable and non-removable memory components, such as RAM, ROM, flash memory, magnetic memory, optical memory, USB memory devices, hard disk memory, external memory, and so forth. In a number of embodiments, the computing device 150 and/or the memory 162 can ICC memory, such as memory provided by a SIM card, a USIM card, a UICC, and so on

A communication module illustrated as transmitter 164 in FIG. 8 may enable computing device 150 to communicate with the heart rate sensor 120 and/or a server device via any suitable wired or wireless communication protocol independently or using I/O circuitry. The wired or wireless network may include a wireless telephony network (e.g., GSM, CDMA, LTE, etc.), one or more standard of the Institute of Electrical and Electronics Engineers (IEEE), such as 802.11 or 802.16 (Wi-Max) standards, Wi-Fi standards promulgated by the Wi-Fi Alliance, Bluetooth standards promulgated by the Bluetooth Special Interest Group, a near field communication standard (e.g., ISO/IEC 18092, standards provided by the NFC Forum, etc.), and so on. Wired communications are also contemplated such as through universal serial bus (USB), Ethernet, serial connections, and so forth.

The computing device 150 may be configured to communicate via one or more networks with a cellular provider and an Internet provider to receive mobile phone service and various content, respectively. Content can comprise map data, which may include route information, web pages, services, music, photographs, video, email service, instant messaging, device drivers, real-time and/or historical weather data, instruction updates, and so forth.

The transmitter 164 can receive control signals and/or other communications from, for example, the heart rate sensor 120. The transmitter 164 can be communicatively coupled to the heart rate sensor 120 via a wired or wireless connection. Accordingly, the transmitter 164 can be a wireless transmitter configured to transmit commands to the heart rate sensor 120 and/or receive data, including heart rate data, from the heart rate sensor 120 via Bluetooth and/or a cellular network, for example.

The computing device 150 may include a number of sensors including a location determining component 166. The location determining component 166 can be a GNSS receiver configured to detect a position measurement for the computing device 150. The computing device 150 can further include a barometric pressure sensor 168 to determine an elevation.

The data from the Internet, the number of sensors of the computing device 150, and the data from the heart rate sensor 120 can be used to determine when to work an equine, how long to work the equine, and/or how hard to work the equine. For example, the computing device 150 may use a temperature from real-time weather data based on the detected position measurement for the computing device 150 and/or a temperature from the equine to determine when a user should start exercising the equine, stop exercising the equine, or blanket the equine.

The computing device 150 can receive data including heart rate data and sensor data from a number of heart rate sensors 120. For example, the computing device 150 can receive data from each of a number of heart rate sensors analogous to heart rate sensor 120 and show biometric data or fitness data for each equine wearing a heart rate sensor 120 via user interface 172. The received data can be used to track and convey performance or status of a number of equines before, during, and/or after engaging in various activities, such as sleeping, walking, trotting, running, and/or jumping. A veterinarian, a stable manager, an equine events judge, or an owner can monitor one or more equines from a remote location, which can provide real-time data and alert them to an equine fouling or suffering from colic or heat exhaustion, for example.

The real-time analysis and integration of equine heart rate, performance metrics, geographic information, and/or other information available to the computing device 150 offers the potential to provide immediate feedback to riders, trainers, and caretakers about the equine's current condition. By utilizing advanced algorithms and machine learning techniques, the computing device 150 can generate real-time alerts or notifications based on predefined thresholds or patterns indicative of potential issues or opportunities for improvement.

For example, if the equine's heart rate exceeds a certain level or remains elevated for an extended period during a training session, the rider could receive an instant notification suggesting a break or a reduction in intensity. Similarly, real-time analysis of accelerometer data combined with GPS information can alert users if the equine demonstrates a sudden change in gait or speed on specific terrains or under particular environmental conditions, allowing for immediate adjustments to optimize performance and minimize the risk of injury.

A user may interact with the displayed data via user interface 172, which may include a “soft” keyboard that is presented on a display (e.g., display 154 in FIG. 7) of the computing device 150, an external hardware keyboard communicating via a wired or a wireless connection (e.g., a Bluetooth keyboard), and/or an external mouse, or any other suitable user-input device or component. The user interface 172 may include or communicate with a microphone capable of receiving voice input from a user as well as the display having a touch input.

Although specific embodiments have been illustrated and described herein, those of ordinary skill in the art will appreciate that an arrangement calculated to achieve the same results can be substituted for the specific embodiments shown. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and methods are used. Therefore, the scope of one or more embodiments of the present disclosure should be determined with reference to the appended claims, along with the full range of equivalents to which such claims are entitled.

As used herein, “a number of” something can refer to one or more of such things. As will be appreciated, elements shown in the various embodiments herein can be added, exchanged, and/or eliminated so as to provide a number of additional embodiments of the present disclosure.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A heart rate monitor device, comprising:
a heart rate sensor configured to contact a tail of an equine, wherein the heart rate sensor comprises:
an optical heart rate module (OHR) configured to:
output light into skin of the equine;
receive reflected light; and
convert the reflected light into electrical signals; and
a transmitter configured to wirelessly transmit heart rate data based on the electrical signals; and
a strap configured to:
removably receive the heart rate sensor; and
removably couple to the tail of the equine. wherein the strap is tapered for a circumference of the tail of the equine decreasing.

2. The heart rate monitor device of claim 1, wherein the strap comprises a hook and loop fastener configured to removably couple the strap to the tail of the equine.

3. The heart rate monitor device of claim 1, wherein the strap is elastic.

4. The heart rate monitor device of claim 1, wherein the strap comprises a cavity configured to receive a portion of a finger or hand.

5. The heart rate monitor device of claim 1, wherein the strap comprises a textured feature configured to prevent the strap from moving down the tail of the equine.

6. The heart rate monitor device of claim 1, wherein the strap includes a number of openings configured to allow movement of vertebrae in the tail of the equine.

7. A heart rate monitor device, comprising:
a heart rate sensor configured to contact a tail of an equine, wherein the heart rate sensor comprises:
a tab; and
an optical heart rate module (OHR) configured to:
output light into skin of the equine;
receive reflected light; and
convert the reflected light into electrical signals;
a transmitter configured to wirelessly transmit heart rate data based on the electrical signals; and
a strap comprising a cavity, wherein the strap is configured to:
removably receive the heart rate sensor comprising the tab in the cavity; and
removably couple to the tail of the equine.

8. The heart rate monitor device of claim 7, wherein the tab consistently orients the heart rate sensor within the strap.

9. The heart rate monitor device of claim 7, wherein the heart rate sensor further comprises a button, wherein the tab prevents the button from being accidentally pressed.

10. The heart rate monitor device of claim 7, wherein the cavity is encased in plastic.

11. The heart rate monitor device of claim 7, wherein the heart rate sensor further comprises a light-emitting diode (LED) configured to emit light to indicate the heart rate sensor is powered on.

12. The heart rate monitor device of claim 7, wherein the heart rate sensor further comprises a processor, a memory, a transmitter, a battery, an inertial sensor, a location determining component, a thermometer, and/or a barometric sensor.

13. The heart rate monitor device of claim 12, wherein the inertial sensor, the location determining component, the thermometer, and/or the barometric sensor are configured to:
generate sensor data; and
wirelessly transmit the sensor data.
